(19) United States
(12) Patent Application Publication
(10) Pub. No.: US 2012/0121521 A1
(43) Pub. Date: May 17, 2012

(54) Cosmetic coating to protect unclothed skin from thermal injury

(75) Inventor: John W. Bulluck, Spicewood, TX (US)

(73) Assignee: Texas Research International, Inc.

(21) Appl. No.: 13/066,845

(22) Filed: Apr. 26, 2011

Related U.S. Application Data
(60) Provisional application No. 61/343,253, filed on Apr. 26, 2010.

(51) Int. Cl. A61K 8/73; A61Q 17/00; A61Q 17/04; A61Q 17/02; A61K 8/81; A61Q 1/02

(52) U.S. Cl. 424/59; 514/57; 424/78.02; 424/63

(57) ABSTRACT

A hydrogel composition and a method for protecting a surface from thermal injury is disclosed. The method comprises applying a hydrogel composition to the surface to protect the skin from exposure to a heat flux, wherein the hydrogel is of a type and applied in an amount effective to reduce heat flux by at least 40 percent when exposed to a heat flux of 40 kW/m². Representative examples of such hydrogels include ionized carboxymethyl cellulose hydrogel, ionized polyacrylate hydrogel, and a polymer formed from acrylic acid and crosslinked with polyalkenyl ethers or divinyl glycol.

This application claims priority to U.S. provisional application Ser. No. 61/343,253, filed Apr. 26, 2010, incorporated by reference herein in its entirety.

Subject to rights of the assignee afforded under a Small Business Innovation Research program, the U.S. Government has a paid-up license in this invention and the right in limited circumstances to require the patent owner to license others on reasonable terms as provided for by the terms of contract number W911QY-09-C-0042 awarded by U.S. Army Natick Soldier Research, Development and Engineering Center.

### BACKGROUND OF INVENTION

This invention pertains to a face coatings, such as camouflage face paint, that protect the skin against thermal injuries (burns).

Lives can be saved and burn damage lessened with these new dermal thermal barrier coatings. Burns are generally prevented by clothing and materials that offer a considerable degree of thermal protection from the high heat fluxes. Heat flux is the heat transfer rate per unit area. The size of a fire can be quantified by its heat flux. Historically, burns have comprised 8% to 10% of casualties sustained in post World War II conflicts, and vary based on the type of weapon employed. Explosive devices directed against both civilian and military targets are frequently used in modern wars or acts of terrorism. Protective fire resistant fibers and fabrics or flame resistant treated clothing are critical for warfighters. However, because of ergonomic considerations select areas of the body including the face and the hands may not be covered during operations. In addition, soldiers won't always wear eye protection because the goggles are too hot and tend to remove gloves because of dexterity limitations. What is desired is a comfortable hypoallergenic heat protective skin paint that reduces the severity of the thermal insult to less than 10 kW/m².

There are no protective face paints available that effectively prevent or mitigate the damage caused to skin as a result of exposure to intense radiant heat, particularly heat from explosive events. Specifically, there are no protective or barrier paints available which prevent burns in a military flash fire or accident scenario. Only two seconds skin exposure at 40 kW/m² will cause second degree burns and less than one second at 80 kW/m². What is actually needed is a comfortable hypoallergenic heat protective skin paint that will reduce severity of the thermal insult to less than 10 kW/m². Flash fire is the primary concern; soldiers need four seconds to get out of a vehicle fire. Any new protective composition also needs to be compatible with current camouflage face paints.

### SUMMARY OF INVENTION

The present inventor sought to use non-toxic widely used cosmetic FDA approved components that can dissipate significant amounts of heat energy for the thermal barrier face paint. Early work concerned incorporating silicone resins, intumescing compounds, and phase changing compounds in the coatings. It was recognized, however, that hydrogels had superior performance in the mass loss calorimeter (MLC), differential scanning calorimetry (DSC), and the limiting oxygen index (LOI), the chief thermal evaluation parameters. Consequently, disclosed herein are new thermally effective hydrogel based burn protective dermal coatings.

Novel hydrogel coatings herein demonstrate improved resistance to high heat fluxes of 40 kW/m². These coatings were developed as clear basecoats that can be used in conjunction with a traditional commercial-off-the-shelf (COTS) camouflage topcoat. Alternatively, these clear hydrogel coatings can be pigmented and utilized as stand alone camouflage coatings. Most natural and some synthetic polymers dissolve in water. Some polymers are initially water soluble but like the hydrogels of interest become water insoluble by ionization. As the content of ionized groups increases the polymer becomes more hydrophilic and absorbs more water swelling forming a gel and then completely finally dissolves. Formation and viscosity of the gel depends on the amount of water or concentration of polymer. During the development of pigmented hydrogel coatings Ferro infrared (IR) reflective camouflage pigments were utilized. To establish the potential of using hydrophilic polymers two hydrogel chemistries were evaluated crosslinked polyacrylate and carboxymethyl cellulose (CMC). CMC structure is based on the β-(1→4)-D-glucopyranose polymer of cellulose. Both classes performed very well in the heat absorption tests. Also, in concert testing of commercial camouflage products was performed.

Thus in one broad respect, this invention is a method for protecting skin from thermal injury, which comprises applying a hydrogel composition to the skin to protect the skin from exposure to a heat flux, wherein the hydrogel is of a type and applied in an amount effective to reduce heat flux by at least 40 percent when exposed to a heat flux of 40 kW/m². As used herein, the term hydrogel refers to a colloidal gel in which water is the dispersion medium and maintains a three dimensional structure. In one embodiment, the hydrogel composition includes camouflage pigment. In another embodiment, the hydrogel composition reduces heat flux from 40 kW/m² to 10 kW/m².

In another broad respect, this invention is a camouflage paint, which comprises hydrogel and camouflage pigment.

In another broad respect, this invention is a method for manufacturing a camouflage paint composition, comprising: combining hydrogel and a camouflage pigment to form a composition is effective to reduce heat flux by at least 40 percent when exposed to a heat flux of 40 kW/m².

The hydrogel compositions of this have several advantages, including but not limited to greatly reduced heat flux, ease of application, are based on off-the-shelf non-toxic components, are economically attractive from a cost perspective, and are comfortable on skin. It is envisioned that this invention can be used by the military, but also can be used by people that might be exposed to extreme heat, such as firefighters, race car drivers, and first responders.

### DETAILED DESCRIPTION OF THE INVENTION

The hydrogels used in the practice of this invention can vary, and include those capable of reducing heat flux by at least 40 percent when exposed to a heat flux of 40 kW/m². In general as used herein, hydrogel refers to a polymeric material that is capable of absorbing more than 20% its weight in water while maintaining a distinct three-dimensional structure. Representative examples of such hydrogels include but are not limited to ionized polyacrylate hydrogel, ionized carboxymethyl cellulose hydrogel, acrylates/beheneth-25 methacrylate copolymer, cross-linked acrylic polymer dispersions, hydrophobically modified, cross-linked polyacrylate powders, and combinations thereof. Preferred hydrogels are ionized carboxymethyl cellulose hydrogel and modified polyacrylates. Representative examples of suitable polymers include CARBOPOL™ type polymers of acrylic acid, crosslinked with polyalkkenyl ethers or divinyl glycol, such as CARBOPOL™, PEMULEN™, and NOVEON™ polymers, which are normally obtained as powders of particles averaging, for example, about 0.2 micron in diameter. Each particle can be viewed as a network structure of polymer chains interconnected by crosslinks. Without the crosslinks, the primary particle would be a collection of linear polymer chains, intertwined but not chemically bonded. These linear polymers are soluble in a polar solvent, such as water. For instance, CARBOPOL™ is a linear polymer and is soluble in water. Other CARBOPOL™ polymers are usually crosslinked and oly swell in water up to 1000 times their original volume (and 10 times their original diameter) to form a gel when exposed to a pH environment above 4-6. The pH of the human skin is typically 5.5. Since the pKa of these polymers is 6±0.5, the carboxylate groups on the polymer backbone ionize upon exposure to water, resulting in repulsion between the negative particles, and this contributes to the swelling. Crosslinked polymers generally do not dissolve in water.

CARBOPOL™ ETD 2623 polymer, a preferred hydrogel of this invention, is a hydrophobically modified, cross-linked polyacrylate powder. CARBOPOL™ Aqua 30 polymer is a cross-linked acrylic polymer dispersion. NOVETHIX™ L-10 polymer is a highly efficient hydrophobically modified alkali-swellable acrylic emulsion (HASE) polymer. All of these polymers exhibited very good water and thermal resistance in our assessments.

Additional polymers that may be suitable include polyalkylene oxides such as PEG and carbonates of PET. Other polymers include hydrophilic polyurethanes soluble in alcohols, dimethylsulfoxide or other physiologically acceptable organic solvents. These hydrophilic polyurethanes exist in a variety of solutions in a variety of solvents, differing only in the molecular weight of the polymeric chain. Once the solvent evaporates, the formed film has controllable affinity for fluids. It also has good physical properties, i.e., the film remains flexible and has a controlled moisture vapor transmission, allowing the evaporation of the perspiration fluid and loss of body heat. These hydrogels based on polyurethanes are a family of hydrophilic polymers which in the presence of hydrogen bonding fluids are converted to hydrogels, each taking up fluids to a predetermined equilibrium level. These soluble polymers form a homogenous gel, cream, or viscous liquid or can be prepared in a form suitable for dispensing from a spray can and can be spread onto the skin surface to evaporate into a solid thin film of polymer coating. These polyurethanes can be available in forms with controllable ethanol/water content and with broad range of mechanical properties.

Included among suitable polymeric compositions are hydrophilic hydrogels, including polymers and copolymers of acrylonitrile and polyvinylacetate, also linear or slightly branched polymers and copolymers of 2-hydroxethylacrylate and methacrylate, hydrophilic polyurethanes D-3, D-4, etc., in suitable solvents. Also included are polymers and copolymers of acrylonitrile, particularly copolymers with other derivatives of acrylic acid, such as acrylamide, N-substituted acrylamide, acrylhydrazide N-substituted acrylhydrazide, glutarimide, vinylsulfonate acid, acrylic acid and its salts; polyvinylacetate, its copolymers and particularly poly (vinylacetate-covinylalcohol); linear or slightly branched polymers and copolymers of 2-hydroxyethyl acrylate and methylacrylate; poly (N vinylliminocarbonyl); and polycondensates and polyadducts, such as poly(oxyethyleneoxy carbonylimino-1,3-phenyleniminocarbonyl); poly(oxy-1,4-phenylensulfonyl-1,4-phenylene); poly(imino(1-oxoundecamethylene); poly(pyromellitio dianhydride-co-aromatic amines), or polymaic acid. Particularly advantageous polymeric compounds are those containing at least 2% nitrile groups, such as the polyacrylonitrile and copolymers of acrylonitrile with various, particularly hydrophilic comonomers. A particularly advantageous acrylonitrile copolymer is produced by the partial acid-catalyzed hydrolysis of a polymer containing at least 85 molar percent acrylonitrile units. Polyacrylonitrile and its copolymers, if coagulated from solution of sufficiently high viscosity, forms pseudo-hydrogels (or “aquagels”). The aquagel contains up to about 75% of water, more usually 30 to 60% of water. The water acts as a plasticizer, even if the polymer itself is non-swellable, for essentially an unlimited time period. Such an aquagel thus formed in tissue is a semirigid material suitable, for example, for facial bone augmentation. Hydrogels suitable for the invention are copolymers containing both hydrophilic and hydrophobic groups, such as vinylacetate-vinylalcohol or acrylonitrile-acrylmide. More particularly suitable are copolymers in which both hydrophobic and hydrophilic groups are organized in continuous sequences, or block copolymers.

The hydrogel coatings of this invention may include pigments that provide camouflage coloration to the coatings. Such pigments are well known and available commercially. Coloration can be advantageous in combat environments. Thus pigments that impart colors such as black, brown, tan, green, and so on can be used. During use, two or more differently colored coatings can be applied to the face, for example, such as a black coating and a green coating. A wide range of colors and combination of colors can therefore be employed. For example, the following commercially available (Sun Chemical) pigments can be used: cosmetic yellow, cosmetic green (hydrous), cosmetic black, titanium oxide (atlas white), cosmetic burnt sienna, and cosmetic green anhydrous. The amount of pigment employed will vary depending on the type of pigment and the desired level of coloration desired. Typically the hydrogel coatings contain from 0.1 to 10 percent by weight of a given pigment. The size of the pigments can vary starting from extremely small micron sized particles. Typically the pigment size is larger, however, for ease of admixing. It is also possible to use dyes in the practice of this invention. The dyes may vary widely and are commercially available.

It should be noted that the addition of pigment, or other additive, can affect the hydrogel coating's ability to reduce thermal injury. Accordingly, the type and amount of pigment can be tested to assure that the coating remains capable of reducing heat flux by at least 40 percent when exposed to a heat flux of 40 kW/m². The testing can be performed by using protocols described in the examples herein.

The hydrogel coatings of this invention can optionally be diluted with aqueous and organic solvents to thereby reduce viscosity and potentially improve application on skin or other surface. Representative solvents include but are not limited to DMSO, water, ethanol, and the like.

The hydrogel compositions of this invention may include other additives, such as but not limited to UV absorbing agents, insect repellants such as DEET, adjuvants, EDTA, pH modifier, electrolytes, humectants, surfactants, wetting agents, fragrances, moisturizers, buffers, preservatives, antioxidants, sodium chloride or other salts, an amine crosslinker, a dispersant, and so on.

Potassium sorbate is a preservative widely in foods and packaged goods including cosmetics. It is particularly effective against fungi and yeast. The acidic nature of the hydrogel skin paints of this invention offers resistance to many types of bacteria. Parabens have also been evaluated as microcides. Hydroxybenzoates or parabens are very effective at very low use levels (i.e., 0.08%) and they have a long history of use in cosmetics. A combination of hydroxybenzoates with different alkyl group lengths pendant to the ester functionality usually provides adequate microbe protection.

Triethanolamine can be used as amine crosslinker in the practice of this invention. Triethanolamine is used in cosmetics such as eyeliners, mascara, eye shadows, blushers, bases and foundations, as well as in fragrances, hair care products, hair dyes, wave sets, sunscreens, skin care, and skin cleansing products.

DISPERBYK 180 is generically an alkylolammonium salt of a block copolymer with acidic groups. DISPERBYK™-180 is preferably used to deflocculate, disperse, and stabilize suspensions of inorganic pigments. It is solvent-free. It reduces millbase viscosity and therefore is suitable for low-VOC and VOC-free systems.

The amount of hydrogel composition applied to a surface such as skin will vary depending on the particular formulation but typically an amount effective to form a layer with a minimal aerial density of 5 mg/cm² is utilized. The heat protection improves as the aerial density is increased. As an example a polyacrylate pigment filled coating was applied at three aerial thicknesses, 51 mg/cm², 102 mg/cm², and 154 mg/cm². The time to second degree burn or 44° C. increases 5 to 9 seconds. An uncoated thermocouple takes approximately 3 seconds to reach 44° C. or the heat required for a second degree basal cell burn. Thus, the response depends on application thickness.

The hydrogel composition can be applied in a number of ways, such as manually using a person's finger, manually using an applicator, by spraying, by injection through an injection device, and so on.

The following examples are representative and not intended to limit the scope of the invention or the claims hereto. Unless otherwise denoted, all percentages are by weight.

Three primary tests were used to analyze the coatings. The crucial test was to coat a shovel thermocouple and expose to a heat flux of 40 kW/m². The representative heat flux of 40 kW/m² was used as the reference threat level; this corresponds to an intermediate intensity battlefield fire scenario that would be expected to result in second degree burns to bare skin after approximately two seconds of exposure. The rise in temperature for the coated thermocouple was recorded as a function of time. The slope of the coated thermocouple was compared with an uncoated shovel thermocouple. The best coating exhibited greater than a 60% reduction in the thermal response using the slope of the heating rate as the metric. The second measurement utilized in thermal characterization was differential scanning calorimetry (DSC) and the heat of fusion for the coating in joules/gram. This a measurement of the heat energy absorbed by the sample when it undergoes thermal transitions. Again, very high values were noted for the hydrogel formulations, over 1,300 joules/G. This was almost twice the value for the best commercial camouflage face paint. Water has a very high thermal capacity. Water also has the highest volumetric heat capacity of all commonly used materials, making it ideal for this application. Another screening test utilized in the project was the limiting oxygen index (LOI) and many of the new formulation had LOIs above 95%, this is better than Teflon™. The LOI indicates the oxygen required for sustained ignition. It is very unusual to find any materials with a LOI over 90%. For example, Kevlar™ has a LOI of 68%. The new hydrogel coatings can be easily removed using soap and water.

Hydrogel coatings were tested to determine their LOI, heat of fusion, and percentage resistance to heat flux. The following table reports the results for four compositions. Formulations 121-3 and 121-4 are both pigmented.

| | | | | Percentage |
|---|---|---|---|---|
| | | | | response of |
| | | | | thermocouple |
| | | | | slope coated |
| | | | | versus uncoated |
| | | | Heat of Fusion | at 40 kW/m² |
| Formulation | Composition | LOI | J/g | (lower is better) |
| 121-1 | Ionized | 99.7 | 1,078 | 48.54 |
| | carboxymethyl | | | |
| | cellulose | | | |
| | hydrogel | | | |
| 121-2 | Ionized | 99.7 | 1,329 | 51.40 |
| | polyacrylate | | | |
| | hydrogel | | | |
| 121-3 | Green | 99.7 | 306 | 55.96 |
| | pigmented | | | |
| | ionized | | | |
| | carboxymethyl | | | |
| | cellulose | | | |
| 121-4 | Green | 99.7 | 1,082 | 60.49 |
| | pigmented | | | |
| | ionized | | | |
| | polyacrylate | | | |
| | hydrogel | | | |

By contrast, several commercially available face paints were tested. The results are shown in the following table.

| | | | Percentage |
|---|---|---|---|
| | | | response of |
| | | | thermocouple |
| | | | slope coated |
| | | | versus uncoated |
| Product and | | Heat of Fusion | at 40 kW/m² |
| manufacturer | LOI | J/g | (lower is better) |
| Iguana Camo Face | 30 | 57.1 | 78.87 |
| Paint with DEET | | | |
| Camo Face Paint | 90 | 132.92 | 81.61 |
| Intra red reflective, | | | |
| B.C.B. Int'l | | | |
| Facepaint Olive | 90 | 90.09 | 82.73 |
| B.C.B. Int'l | | | |
| Carbomask Green, | 95 | 976.7 | 71.70 |
| Hutton Labs | | | |
| Paint Face | 95 | 75.48 | 79.16 |
| Camouflage, RJS | | | |
| Scientific | | | |
| Compact, RJS | 60 | 115.61 | 83.18 |
| Scientific | | | |
| Carbomask Black, | 95 | 830.12 | 77.05 |
| Hutton Labs | | | |

The hydrogel coatings in the first table for formulations 121-1, 121-2, 121-3, and 121-4 show significantly improved thermal protection when compared to the commercially available products. The hydrogel formulations of this invention have a percentage response of thermocouple slope coated versus uncoated at 40 kW/m² of less than 70%, preferably less than 65%, and for unpigmented compositions less than 55%.

For field trials 15 gallons of green skin cream and fifteen gallons of green skin cream with DEET was prepared. The following steps were followed in the preparation of the skin cream materials.
1. A-Component. High speed dispersion of the pigments and dispersant in 25% of water at 3500 rpm until a Hegman grind value of 8 is achieved. Add remaining water and let down for thirty minutes, 800 rpm.—set aside A component
2. B-Component. Use a Hobart or low speed mixer for B component. Heat water to 40° C. and dissolve potassium sorbate. Add the Carbopol ETD2623 and mix until a stiff hydrogel produced then add pigment dispersion component A and mix at low speed. For the DEET containing compositions the DEET is added immediately after the stiff hydrogel is produced and mixed for thirty minutes, and then the pigment dispersion is added. After it is well mixed add triethanolamine crosslinker and mix at low speed for thirty minutes, dispense into containers.

### Typical Composition

### A-Component Grind

1. Water—47.6%
2. FDA Approved Inorganic Cosmetic Pigment Blend—47.7%
3. DISPERBYK™ 180—4.7%-

### Typical B-Component Low Shear Mixing

1. Water=53.97%
2. Potassium Sorbate=0.11%
3. CARBOPOL™ ETD 2623=1.01%
4. Component A (above)=44.56%
5. Triethanolamine=0.35%

The hydrogel composition was dispensed in two ounce tubes.

Testing was also performed to insure that the skin coatings were non-irritating or hypoallergenic. The purpose of the Epiderm skin testing is to evaluate the potential dermal irritancy of a challenge compound to the EpiDerm construct (MatTek Corporation) as determined by the exposure time of a test article required to reduce cell viability to 50% of control viability. This cell viability is measured by the NAD(P)H-dependent microsomal enzyme reduction of MIT to a blue formazan precipitate, in treated cultures, and is expressed as a percentage relative to untreated (negative control) cultures. The hydrogel coatings of this invention were found to be non-irritating.

Further modifications and alternative embodiments of this invention will be apparent to those skilled in the art in view of this description. Accordingly, this description is to be construed as illustrative only and is for the purpose of teaching those skilled in the art the manner of carrying out the invention. It is to be understood that the forms of the invention herein shown and described are to be taken as illustrative embodiments. Equivalent elements or materials may be substituted for those illustrated and described herein, and certain features of the invention may be utilized independently of the use of other features, all as would be apparent to one skilled in the art after having the benefit of this description of the invention. In addition, various materials and methods can be excluded from this invention.

What is claimed is:

1. A method for protecting a surface from thermal injury, comprising: applying a hydrogel composition to the surface to protect the skin from exposure to a heat flux, wherein the hydrogel is of a type and applied in an amount effective to reduce heat flux by at least 40 percent when exposed to a heat flux of 40 kW/m².

2. The method of claim 1, wherein the surface is human skin.

3. The method of claim 1, wherein the surface is skin of a human face or hands.

4. The method of claim 1, wherein the hydrogel composition includes camouflage pigment.

5. The method of claim 1, wherein the hydrogel is an ionized carboxymethyl cellulose hydrogel.

6. The method of claim 1, wherein the hydrogel is an ionized polyacrylate hydrogel.

7. The method of claim 1, wherein the hydrogel is a polymer formed from acrylic acid and crosslinked with polyalkenyl ethers or divinyl glycol.

8. The method of claim 1, wherein the hydrogel composition includes a UV absorbing agent, an insect repellant, an adjuvant, EDTA, a pH modifier, an electrolyte, a humectant, a surfactant, a wetting agent, a fragrance, a moisturizer, a buffer, a preservative, an amine crosslinker, a dispersant, an antioxidant, sodium chloride, or a combination thereof.

9. The method of claim 1, wherein the hydrogel composition includes triethanolamine, potassium sorbate, and a dispersant.

10. The method of claim 1, wherein the hydrogel composition is made by admixing an A-component and a B-component, where the A component is prepared by mixing water, a pigment, and a dispersant, and the B-component is prepared by mixing water, potassium sorbate, a hydrogel polymer, triethanolamine, and the A-component.

11. The method of claim 1, wherein the hydrogel composition is applied from a storage container.

12. A skin paint composition, comprising: a hydrogel and camouflage pigment. wherein the hydrogel is of a type and applied in an amount effective to reduce heat flux by at least 40 percent when exposed to a heat flux of 40 kW/m².

13. The skin paint composition of claim 12, wherein the hydrogel is an ionized carboxymethyl cellulose hydrogel, an ionized polyacrylate hydrogel, or a polymer formed from acrylic acid and crosslinked with polyalkenyl ethers or divinyl glycol.

14. The skin paint composition of claim 12, wherein the hydrogel composition includes a UV absorbing agent, an insect repellant, an adjuvant, EDTA, a pH modifier, an electrolyte, a humectant, a surfactant, a wetting agent, a fragrance, a moisturizer, a buffer, a preservative, an amine crosslinker, a dispersant, an antioxidant, sodium chloride, or a combination thereof.

15. The skin paint composition of claim 12, wherein the hydrogel composition includes triethanolamine, potassium sorbate, and a dispersant.

16. A method for manufacturing a skin paint composition, comprising: combining hydrogel and a pigment to form a composition is effective to reduce heat flux by at least 40 percent when exposed to a heat flux of 40 kW/m².

17. The method of claim 16, further comprising filling a storage container with the skin paint composition and sealing the container.

18. The method of claim 16, wherein the hydrogel is an ionized carboxymethyl cellulose hydrogel, an ionized polyacrylate hydrogel, or a polymer formed from acrylic acid and crosslinked with polyalkenyl ethers or divinyl glycol.

19. The method of claim 16, wherein the skin paint composition includes a UV absorbing agent, an insect repellant, an adjuvant, EDTA, a pH modifier, an electrolyte, a humectant, a surfactant, a wetting agent, a fragrance, a moisturizer, a buffer, a preservative, an amine crosslinker, a dispersant, an antioxidant, sodium chloride, or a combination thereof.

20. The method of claim 16, wherein the skin paint composition includes triethanolamine, potassium sorbate, and a dispersant.

21. The method of claim 16, wherein the skin paint composition is made by admixing an A-component and a B-component, where the A component is prepared by mixing water, a pigment, and a dispersant, and the B-component is prepared by mixing water, potassium sorbate, a hydrogel polymer, triethanolamine, and the A-component.
